(19) United States
(12) Patent Application Publication
(10) Pub. No.: US 2003/0212134 A1
(43) Pub. Date: Nov. 13, 2003

(54) Combination of pinitol and creatine to enhance uptake and retention of creatine

(76) Inventor: John C. Dykstra, Eden Prairie, MN (US)

Correspondence Address: SHERRILL LAW OFFICES, 4756 BANNING AVE, SUITE 212, WHITE BEAR LAKE, MN, 55110-3205, US

(21) Appl. No.: 10/240,611

(22) PCT Filed: Apr. 16, 2001
(86) PCT No.: PCT/US01/12485

(51) Int. Cl. A61K 31/70; A61K 31/198; A61K 31/045

(52) U.S. Cl. 514/565; 514/23; 514/729

(57) ABSTRACT

Muscle performance can be improved and muscle hypertrophy enhanced, through enhanced uptake and retention of creatine resulting in improved ATP resynthesis, by administering an effective synergistic amount of a combination of creatine or an active derivative thereof and pinitol or an active derivative or metabolite thereof.

### FIELD OF THE INVENTION

The invention relates to the use of pharmaceuticals and dietary supplements to enhance the uptake and retention of creatine in muscle, tissue.

### BACKGROUND

Creatine phosphate is an excellent source of immediately available energy, capable of fueling the resynthesis of ATP from ADP.

Creatine is not synthesized in muscle tissue, but is transported to muscle tissue by the blood stream. Mankind has sought ways to accelerate ATP resynthesis and improve muscle power and performance by enhancing creatine uptake and retention by the muscle tissue. Such efforts range from the consumption of mass quantities of creatine to the administration of a combination of creatine and any of a variety of adjuvants, such as insulin and/or a carbohydrate.

While certain of these techniques have demonstrated limited success in enhancing creatine uptake and/or retention in muscle tissues, the search continues for safe alternative means for improving muscle performance by enhancing creatine uptake and retention in muscle tissue.

### SUMMARY OF THE INVENTION

We have discovered that muscle performance can be improved and muscle hypertrophy enhanced, through enhanced uptake and retention of creatine resulting in improved ATP resynthesis, by administering an effective synergistic amount of a combination of creatine or an active derivative thereof and pinitol or an active derivative or metabolite thereof.

### DETAILED DESCRIPTION OF THE INVENTION INCLUDING A BEST MODE

The First Active Ingredient

The first active ingredient is creatine or an active derivative thereof. Creatine has the structure shown below in FIG. 1.

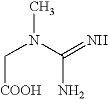

Creatine is commercially isolated from meat extracts and is available from a number of sources. Creatine is generally available and suitable for use in the invention as a monohydrate, with the monohydrate converted to anhydrous form at about 100° C.

Suitable derivatives of creatine include creatine phosphates, creatine citrate, creatine ascorbate, creatine pyruvate, cyclo-creatine and other salts thereof

The Second Active Ingredient

The second active ingredient is pinitol or an active derivative or metabolite thereof Pinitol has the structure shown below in FIG. 2.

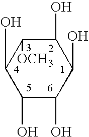

Pinitol can be extracted from a number of plant sources, including alfalfa, Bougainvillea leaves, chick peas, pine trees and soy beans, and is commercially available from Humanetics Corporation under the trademark Inzitol™.

Suitable derivatives and metabolites of pinitol include pinitol glycosides, pinitol phospholipids, esterified pinitol, lipid-bound pinitol, pinitol phosphates, pinitol phytates, and hydrolyzed pinitol such as d-chiro-inositol.

Optional Third Active Ingredient

The composition can optionally include a third active ingredient of a carbohydrate or an active derivative thereof (i.e., an organic aldehyde or ketone compound having multiple hydroxyl groups). The carbohydrate is preferably a simple carbohydrate, such as fructose or glucose.

Administration

Administration Route

The active ingredients can be administered to any mammal, including humans, for whom an improvement in muscle performance and/or enhanced muscle hypertrophy is desired.

The active ingredients can be administered individually or together by virtually any of the commonly accepted practices for the administration of pharmaceutical preparations and dietary supplements including specifically, but not exclusively, mucosal administration, oral consumption, ocular administration, subcutaneous injection, transdermal administration, etc. In order to achieve the desired synergistic effect, the creatine and pinitol should be administered so that the blood plasma level of pinitol is elevated simultaneously with or prior to administration of creatine. In a preferred embodiment, the creatine and pinitol are blended together in a single composition and administered together.

Mucosal administration of the active ingredients includes such routes as buccal, endotracheal, nasal, pharyngeal, rectal, sublingual, vaginal, etc. For administration through the buccal/sublingual/pharyngeal/endotracheal mucosa, the ingredients may be formulated as an emulsion, gum, lozenge, spray, tablet or an inclusion complex such as cyclodextrin inclusion complexes. Nasal administration is conveniently conducted through the use of a sniffing power or nasal spray. For rectal and vaginal administration, the ingredients may be formulated as a cream, douche, enema or suppository.

Oral consumption of the active ingredients may be effected by incorporating the ingredients into a food or drink, formulating the ingredients into a chewable or swallowable tablet, or formulating the ingredients as a powdered or granular dietary supplement.

Ocular administration may be effected by incorporating the active ingredients into a solution or suspension adapted for ocular application such as drops or sprays.

Subcutaneous administration involves incorporating the active ingredients into a pharmaceutically acceptable and injectable carrier.

For transdermal administration, the active ingredients may be conveniently incorporated into a lipophilic carrier and formulated as a topical creme or adhesive patch.

Dose Rate

The range of dosages and dose rates effective for achieving the desired synergistic improvement in muscle performance and enhanced muscle hypertrophy may be determined in accordance with standard industry practices. As a general guide, a dose rate of about 5 to 20 grams of creatine per day, accompanied by appropriate amounts of pinitol and any carbohydrate, should provide the desired biological response Of improved muscle performance and enhanced muscle hypertrophy.

Administration can be effected on any desired schedule with a preference for administration with every meal, daily or a few hours prior to each workout or other athletic activity.

Ratio of First Ingredient to Second Ingredient

The range of relative percentages of creatine and pinitol (i e., the active ingredients) effective for achieving the desired synergistic improvement in muscle performance and enhanced muscle hypertrophy may similarly be determined in accordance with standard industry practices. As a general guide, a weight ratio of pinitol or active derivative or metabolite thereof to creatine or active derivative thereof between 1:10,000 to 1:1 should provide the desired biological response of improved muscle performance and enhanced muscle hypertrophy. Preferred ratios are between about 1:200 (e.g, 100 mg of pinitol to 20 grams of creatine) to about 1:2 (e.g., 1 g of pinitol to 2 grams of creatine).

I claim:

1. A composition, comprising:
(a) pinitol or an active derivative or metabolite thereof and
(b) creatine or an active derivative thereof.

2. The composition of claim 1 wherein the creatine is creatine monohydrate.

3. The composition of claim 1 wherein the weight ratio of pinitol or active derivative or metabolite thereof to creatine or active derivative thereof is between 1:10,000 to 1:1.

4. The composition of claim 1 wherein the composition is in powder or granular form.

5. The composition of claim 1 wherein the composition is in tablet form.

6. The composition of claim 1 wherein the composition further includes a carbohydrate or an active derivative thereof.

7. A method of increasing creatine retention in a mammal comprising substantially simultaneously increasing the blood plasma level of creatine and pinitol.

8. The method of claim 7 wherein the mammal is a human.

9. The method of claim 7 wherein the blood plasma level of creatine is increased by the administration of creatine.

10. The method of claim 9 wherein the administration of creatine comprises oral consumption of creatine.

11. The method of claim 10 wherein the creatine is creatine monohydrate.

12. The method of claim 7 wherein the blood plasma level of pinitol is increased by the administration of pinitol.

13. The method of claim 12 wherein the administration of pinitol comprises oral consumption of pinitol.

14. The method of claim 10 wherein the blood plasma level of pinitol is increased by the oral consumption of pinitol.

15. The method of claim 14 wherein the weight ratio of pinitol to creatine is between 1:10,000 to 1:1.

16. The method of claim 14 wherein the composition is in powder or granular form.

17. The method of claim 14 wherein the composition is in tablet form.

18. The method of claim 14 wherein the composition is consumed daily.

19. The method of claim 14 wherein at least 5 grams of creatine and at least 2 mg of pinitol are consumed daily.

20. The method of claim 19 wherein at least 5 grams of creatine and at least 10 mg of pinitol are consumed daily.

21. A method of increasing cell up-take of creatine in a mammal comprising the substantially simultaneous administration of creatine or active derivative thereof and pinitol or active derivative or metabolite thereof.

22. The method of claim 21 wherein the mammal is a human.

23. The method of claim 22 wherein the administration of creatine comprises oral consumption of creatine.

24. The method of claim 23 wherein the creatine is creatine monohydrate.

25. The method of claim 22 wherein the administration of pinitol comprises oral consumption of pinitol.

26. The method of claim 25 wherein the administration of creatine comprises oral consumption of creatine.

27. The method of claim 26 wherein the weight ratio of pinitol to creatine is between 1:10,000 to 1:1.

28. The method of claim 27 wherein the composition is in powder or granular form.

29. The method of claim 27 wherein the composition is in tablet form.

30. The method of claim 27 wherein the composition is consumed daily.

31. The method of claim 27 wherein at least 2 mg of pinitol and at least 5 grams of creatine are consumed daily.

32. The method of claim 31 wherein at least 10 mg of pinitol and at least 5 grams of creatine are consumed daily.

33. The method of claim 22 wherein the method of increasing cell up-take of creatine comprises increasing muscle cell up-take of creatine.

34. A method of accelerating ATP resynthesis in a mammal comprising the substantially simultaneous administration of creatine or active derivative thereof and pinitol or active derivative or metabolite thereof.

35. The method of claim 34 wherein the mammal is a human.

36. The method of claim 35 wherein the administration of creatine comprises oral consumption of creatine.

37. The method of claim 36 wherein the creatine is creatine monohydrate.

38. The method of claim 35 wherein the administration of pinitol comprises oral consumption of pinitol.

39. The method of claim 38 wherein the administration of creatine comprises oral consumption of creatine.

40. The method of claim 39 wherein the weight ratio of pinitol to creatine is between 1:10,000 to 1:1.

41. The method of claim 40 wherein the composition is in powder or granular form.

42. The method of claim 40 wherein the composition is in tablet form.

43. The method of claim 40 wherein the composition is consumed daily.

44. The method of claim 40 wherein at least 2 mg of pinitol and at least 5 grams of creatine are consumed daily.

45. The method of claim 44 wherein at least 10 mg of pinitol and at least 5 grams of creatine are consumed daily.

46. The method of claim 22 wherein the method of accelerating the rate of ATP resynthesis comprises accelerating the rate of ATP resynthesis in muscle cells.

47. A method of increasing muscle hypertrophy in a mammal comprising the substantially simultaneous administration of creatine or active derivative thereof and pinitol or active derivative or metabolite thereof.

48. The method of claim 47 wherein the mammal is a human.

49. The method of claim 48 wherein the administration of creatine comprises oral consumption of creatine.

50. The method of claim 49 wherein the creatine is creatine monohydrate.

51. The method of claim 48 wherein the administration of pinitol comprises oral consumption of pinitol.

52. The method of claim 51 wherein the administration of creatine comprises oral consumption of creatine.

53. The method of claim 52 wherein the weight ratio of pinitol to creatine is between 1:10,000 to 1:1.

54. The method of claim 53 wherein the composition is in powder or granular form.

55. The method of claim 53 wherein the composition is in tablet form.

56. The method of claim 53 wherein the composition is consumed daily.

57. The method of claim 53 wherein at least 2 mg of pinitol and at least 5 grams of creatine are consumed daily.

58. The method of claim 57 wherein at least 10 mg of pinitol and at least 5 grams of creatine are consumed daily.
